(19) United States
(12) Patent Application Publication
(10) Pub. No.: US 2007/0154437 A1
(43) Pub. Date: Jul. 5, 2007

(54) Peptide-tagged proteins and methods of making and using thereof

(76) Inventors: Pingfan Rao, Fuzhou (CN); Shutao Liu, Fuzhou (CN)

Correspondence Address: DLA PIPER US LLP, 1200 Nineteenth Street, N.W., Washington, DC, 20036-2412, US

(21) Appl. No.: 11/604,818

(22) Filed: Nov. 28, 2006

Related U.S. Application Data
(63) Continuation-in-part of application No. 10/232,410, filed on Sep. 3, 2002.

(51) Int. Cl. A61K 8/65; A61K 38/44

(52) U.S. Cl. 424/70.14; 424/94.4

(57) ABSTRACT

A method for the treatment of a skin or hair condition or for the alteration of a physical feature of the hair and skin is disclosed. The method utilizes a fusion protein comprising a peptide having SOD activity and a membrane transport sequence for the treatment of skin and hair conditions such as wrinkles, pigmentation, skin burn and hair loss. A composition comprising the fusion protein and a carrier is also disclosed.

This application is a continuation-in-part application of U.S. patent application Ser. No. 10/232,410, filed Sep. 3, 2002. The entirety of the U.S. patent application Ser. No. 10/232,410 is incorporated herein by reference.

### FIELD

The present invention relates to the field of molecular biology, cell biology and dermatology. Particularly, the present invention relates to fusion proteins and the use of protein signal sequences to adapt the delivery of an enzyme or enzyme inhibitor to enhance the treatment or alteration of physical features of the skin and hair.

### BACKGROUND

Topical application of active agents in affecting hair and skin conditions is well known. However, topical application of active agents for intracellular delivery across the cellular membrane for intra-cellular activity has been less evident.

Currently, intracellular delivery is accomplished by utilizing viral vectors or non-viral delivery strategies. Historically, non-viral delivery strategies have not been efficient for delivering macromolecules across the cell membrane when compared with viral vectors. However, delivery of the active agent across the membrane to enable access to particular intracellular regions of the cell has proven meaningful in modulating cellular activity.

Physical features of the hair and skin reflect the condition of the cells in the dermal and epidennal layers of the skin, including constituent cells such as keratinocytes, follicle cells, melanocytes, adipose cells and others. The regulation of the enzymatic activities within certain cells can offset deterioration in both hair and skin features. Enzymatic regulation is accomplished by increasing enzyme content or activity to offset any deficit, or by enzymatic inhibition to down-regulate activity along a particular enzymatic pathway. Alterations in cellular enzyme activity can lead to desirable changes in both hair and skin features.

The skin is a versatile organ that serves as a self-renewing and self-repairing interface between the body of a vertebrate organism and its environment, and covers almost the entire external surface of the body. The skin is continuous with, but distinct from, the mucosae of the alimentary, respiratory, and urogenital tracts. The specialized skin of the mucocutaneous junctions connects the skin and the mucosae.

Skin can be divided into two major classes: thin, hairy (hirsute) skin which covers most of the body, and thick, hairless (glabrous) skin which forms the surfaces of the palm of the hands, sole of the feet, and flexor surface of the digits. Both classes of skin are composed of three basic layers: the epidermis, the dermis, and the hypodermis. The primary differences in the two classes of skin are in the thickness of their epidermal and dermal components, and in the presence of hairs with their attendant sebaceous glands and arrector pili muscles (pilasebaceous units).

The epidermis, a stratified keratinous squamous epithelium is primarily composed of keratinocytes and can be further divided into several strata (from deep to superficial): stratum basale, stratum spinosum, stratum granulosum, stratum lucidum, and stratum corneum. Epidermal appendages, such as pilosebaceous units, sudoriferous gland, and nails are formed by ingrowth or other modifications of the general epidermis, which is often referred to as the interfollicular epidermis.

In addition to keratinocytes, the mature epidermis also contains nonkeratinocytes including melanocytes which are pigment-forming cells, Langerhans cells which are immunocompetent antigen-presenting cells derived from bone marrow, and lymphocytes. The epidermis also includes Merkel cells, which are modified keratinocytes.

The skin mediates a variety of important local and systemic functions, including maintenance of skin texture, skin color and hair color. These normal skin cell functions can be exploited to modulate the enzymatic pathway associated with lipid generation, hair pigmentation, and removal of free radicals formed as a result of UV exposure.

The skin is an attractive target organ due to its accessibility, thereby providing one of the easiest routes of administration. Moreover, because it is a stratified epithelium, skin allows for the possibility of targeting either differentiated or proliferative cells, depending upon the desired effect of the active agent. In addition, epidermal biology is relatively well-characterized at both the cellular and molecular levels.

It has proven difficult to develop effective methods for importing biologically active molecules into cells, both in vivo and in vitro. Crossing the lipid bilayer has proven to be a significant impediment and no effective means has been developed for the topical application of agents affecting the enzymatic pathways in skin cells. A solution to this problem would greatly expand treatments to skin and hair conditions for which delivery of a biologically active agent to the cell interior would benefit.

In general, conventional non-invasive methods involve pretreatment of the skin to remove hair. However, the more complicated the delivery method or the delivery formulation, the more difficult it is to apply these methods and formulations in the field. Methods that use needles or require multiple dosages via an invasive route meet with problems of patient compliance. In the case of intracellular delivery, it would be desirable to have a means to avoid the use of virus delivery vehicles, which may have undesirable side effects and safety concerns.

Few drugs readily penetrate the intact skin. There is a need in the field for methods of delivery of active agent proteins to within skin cells that does not require special formulations or invasive procedures to facilitate delivery of the protein into skin cells.

### SUMMARY

One aspect of the present invention relates to a method for treating a skin condition or alteration of a physical feature of the hair or skin in a mammal. The method comprises the steps of applying to a skin area of a mammal in need of such treatment, a composition comprising an effective amount of a fusion protein and a pharmaceutically acceptable carrier. The fusion protein comprises a peptide having superoxide dismutase (SOD) activity and a membrane transport sequence.

In one embodiment, the method is used for skin depigmentation. In another embodiment, the method is used for wrinkle removal. In another embodiment, the method is used for skin whitening. In another embodiment, the method is used for treating skin burn, such as sunburn. In another embodiment, the method is used for treating hair loss.

In another embodiment, the carrier comprises water, glyceryl stearate, cetyl alcohol, propylene glycol stearate, polysorbate 60, and sorbitan stearate, Vitamin E, methylparaben, propylparaben, and BHA.

In another embodiment, the membrane transport sequence is selected from the group consisting of: SEQ ID NOs: 1-13 and a polylysine.

In yet another embodiment, the membrane transport sequence is a membrane transport sequence from HIV Tat protein.

Another aspect of the present invention relates to a composition for skin treatment. The composition comprises a fusion protein having SOD activity, and a carrier.

In one embodiment, the fusion protein comprises a peptide having SOD activity and a membrane transport sequence.

In another embodiment, the membrane transport sequence is a membrane transport sequence from HIV Tat protein.

Other features and advantages of the present invention will be readily appreciated as the same becomes better understood by reference to the following detailed description.

### BRIEF DESCRIPTION OF THE FIGURES

FIG. 1 is a schematic showing that HIV Tat membrane transport sequence facilitates the entry of a fusion protein containing such sequence into a cell.

FIG. 2 is a composite of pictures showing that Tat-eGFP fusion protein is internalized into human hepatic cells, human hepatic carcinoma cells, and human cervical carcinoma cells. Panel A, white light; Panel B, green fluorescence; Panel C, overlap of A and B. SMMC-7721: human hepatic carcinoma cell line; BEL-7402: human hepatic carcinoma cell line; L02: human normal hepatic cell line; Hela: human cervical carcinoma cell line. Microscopy study: x400; Fluorescence: Em=484nm, Ex=510 nm; Zeiss Axiophot. Cell Culture: 10% Fetal Bovine Serum in DMEM or RPM11640, 37° C., 5% CO₂. After 24 hours cultivation, cells were incubated with Tat-eGFP (4 μmol/L) for 6 hours.

FIG. 3 is a composite of pictures showing that eGFP control protein is not internalized into human hepatic cells, human hepatic carcinoma cells, and human cervical carcinoma cells. Panel A, bright light; Panel B, green light; Panel C, overlap of A and B. Experiment conditions were the same as described in FIG. 2.

FIG. 4 is a composite of pictures showing the concentration-dependent transduction of Tat-eGFP through the membrane of Hela cells. Panel A, phase contrast images (white light); Panel B, fluorescence images (green fluorescence), Panel “Merge,” an overlap of the white light and green fluorescence. Row A, Tat-eGFP fusion protein at 1 μmol/L; Row B, Tat-eGFP fusion protein at 2 μmol/L; Row C, Tat-eGFP fusion protein at 4 μmol/L; Row D, control group with eGFP at 4 μmol/L. Microscopy study: x400; Fluorescence: Em=484nm, Ex=510 nm; Zeiss Axiophot. Cell Culture: 10% Fetal Bovine Serum in DMEM or RPM11640, 37° C., 5% CO2. After 24 hours cultivation, cells were incubated with proteins for 6 hours.

FIG. 5 is a composite of pictures showing the concentration-dependent transduction of Tat-eGFP through the skin of C. elegan. C. elegan was incubated at 20° C. with the fusion protein Tat-eGFP at 3 μM, 6 μM, 12 μM respectively. After 1, 3 and 6 hours incubation, C. elegan was recovered, and then, washed with PBS buffer to remove Tat-eGFP on its surface and than observed under fluorescent microscope.

FIG. 6 is composite of pictures showing the delivery of Tat-GFP into mouse skin through hair follicles. Left panel (FIG. 6A): Phase contrast image (bright light). Right panel (FIG. 6B): Fluorescence image (green light).

FIG. 7 is a diagram showing internalization of Tat-SOD into cells. Hela cells were co-cultured with 500 μg/mL SOD or PTD-SOD in fresh RPMI 1640 medium (PBS buffer was used as control). After 4 hours, the total SOD activity of cell lysate was assayed spectrophotometrically at 505 nm with a commercial kit of xanthine-xanthine oxidase system (Cat. No. A001 from Jian Cheng Institute of Biotechnology, Nanjing, China). Data shown was the average ±s.d. of 4 measurements.

FIG. 8 is a diagram showing internalization of Tat-SOD at various concentrations. Hela cells co-cultured with PTD-SOD of different concentration in fresh RPMI 1640 medium (PBS buffer was used as control). After 4 hours, the total SOD activity of cell lysate was assayed spectrophotometrically at 505 nm with a commercial kit of xanthine-xanthine oxidase system (Cat. No. A001 from Jian Cheng Institute of Biotechnology, Nanjing, China). Data shown was the means of 3 measurements.

FIG. 9 is a diagram showing internalization of Tat-SOD into mitochondria. Mitochondria from rat liver cells were incubated with 500 μg/mL TAT-SOD in fresh RPMI 1640 medium (PBS buffer was used as control) for 0.5, 1.5, 2.5, 3.5, or 4.5 hours. Trypsin was added to remove the PTD-SOD attached on mitochondria surface, and the total SOD activity of mitochondria lysate was assayed spectrophotometrically at 505 nm with a commercial kit of xanthine-xanthine oxidase system (Cat. No. A001 from Jian Cheng Institute of Biotechnology, Nanjing, China). Data shown was the means of 3 measurements.

FIG. 10 is a diagram showing Tat-SOD-mediated recovery in cells damaged by alloxan. MDCK cells (Madin-Darby canine kidney cell line) were inoculated to 96-well culture plate and incubated overnight. 10 umol/L Alloxan was added into the damaged group to damage the cell for an hour before the addition of the culture media, while SOD group was added with the media with SOD (final concentration 6 mg/ml), and the sample group was added with the media with TAT-SOD (0.05, 0.1, 0.2, 0.4 and 0.6 mg/ml). All groups were incubated for 24 hours, and measured with MTT method at 590 nm. Bar number 1, 2, 3, 4, 5 represent TAT-SOD concentrations of 0.05, 0.1, 0.2, 0.4 and 0.6 mg/ml, respectively. Normal group represent cells without damage by Alloxan. In comparison with the damaged group, * represents P<0.05, ** represents P<0.01.

FIG. 11 is a diagram showing the wrinkle removal effect of Tat-SOD. The change of total length of wrinkles shown as the delta wrinkle length (in mm). Delta wrinkle length at each visit was significantly different from each other (P<0.05, n=15).

FIG. 12 is a diagram showing the skin whitening effect of Tat-SOD. Whitening effect (L value change) was determined at indicated days after the start of topical applications of Tat-SOD cream. Control: no Tat-SOD cream treatment. p<0.05, n=10.

FIGS. 13A and 13B are pictures showing localized hyperpigment before and after topical application of Tat-SOD, respectively.

FIG. 14 is a diagram showing that Tat-SOD reduces erythema index variation (ΔEI) over time.

FIG. 15 is a diagram showing that Tat-SOD reduces skin temperature in sunburn area.

FIG. 16 is a diagram showing that Tat-SOD stopped the development of blister after scalding by boiling oil.

### DETAILED DESCRIPTION

The practice of the embodiments described in further detail below will employ, unless other wise indicated, conventional methods of microbiology, molecular biology, and immunology within the skill of the art. Such techniques are explained fully in the literature. All publications, patents and patent applications cited herein, whether supra or infra, are hereby incorporated by reference in their entirety.

One aspect of the present invention related to methods for making and using a fusion protein composition for preventing or treating skin or hair deficiencies or for altering a physical feature of the hair or skin in a mammal; preferably a human. The present invention offers improved efficacy of delivery of the active agent protein to the cell via a membrane transport sequence.

In describing the present invention, the following terms will be employed, and are intended to be defined as indicated below.

The term “peptide” is used herein interchangeably with “oligopeptide” to designate a series of monomers or residues, typically L-amino acids, connected one to the other typically by peptide bonds between the alpha-amino and carbonyl groups of adjacent amino acids. The term peptide encompasses an isolated or recombinant sequence of amino acids, which may be naturally occurring or non-naturally occurring, and synthetic derivatives or analogues thereof. Sequences of naturally occurring amino acids recited herein utilize the standard amino acid nomenclature using single letter abbreviations for each residue—Alanine (A), Arginine (R), Asparagine (N), Aspartic acid (D), Cysteine (C), Glutamine (Q), Glutamic acid (E), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y), Valine (V). Amino acid “analogues” encompass functionally equivalent modified amino acid residues which are known in the art (see, e.g., U.S. Pat. Nos. 5,221,665 and 6,171,589, both incorporated by reference).

The term “membrane transport sequence” or “MTS” is used to indicate a peptide, or derivative thereof, that directs the transport of a peptide, protein, or molecule associated with the MTS; from the outside of a cell into the cytoplasm of the cell through a cytoplasmic membrane of the cell. Furthermore, a peptide that contains a “membrane transport sequence” and additional amino acid sequences could be used as a “membrane transport sequence” for the purposes of the present invention. An MTS may be composed of D- or L-amino acids.

The term “nuclear localization sequence” or “NLS” is used to indicate a peptide, or derivative thereof, that directs the transport of a peptide, protein, or molecule associated with the NLS; from the cytoplasm into the nucleus of the cell across the nuclear membrane. Furthermore, a peptide that contains a “nuclear localization sequence” and additional amino acid sequences could be used as a “nuclear localization sequence” for the purposes of the present invention Adam et al. (1990) J. Cell. Biol. 111:807-818). In certain embodiments, an NLS may be composed of D- or L-amino acids.

Hair and Skin Color

Melanogenesis is the process of production and subsequent distribution of melanin by melanocytes within the skin and hair follicles. Melanocytes have specialized lysosome-like organelles, termed melanosomes, which contain several enzymes that mediate the production of melanin. The copper-containing enzyme tyrosinase catalyzes the oxidation of the amino acid tyrosine into DOPA, and subsequently, DOPA-quinone. At least two additional melanosomal enzymes are involved in the eumelanogenesis pathway that produces brown and black pigments, including TRP-1 (DHICA oxidase), and TRP-2 (DOPAchrome tautomerase). Depending on the incorporation of a sulfur-containing reactant, such as cysteine or glutathione, into the products, the melanogenesis pathway diverges to produce the pheomelanins of amber and red pigments.

The perceived color of skin and hair is determined in part by the ratio of eumelanins to pheomelanins, and in part by blood within the dermis. The balance in skin hue is genetically regulated by many factors, including but not limited to: (a) the levels of expression of tyrosinase, TRP-2, and TRP-1; (b) thiol conjugation (e.g., with glutathione or cysteine) leading to the formation of pheomelanins; (c) the α-melanocyte-stimulating hormone (α-MSH) and melanocortin receptor, which is coupled to the adenylate cyclase/protein kinase A pathway; (d) the product of the agouti locus, agouti signal protein, which down-regulates pigmentation of hair melanocytes; and (e) yet unknown mechanisms that regulate the uptake and distribution of melanosomes in recipient epidermal and hair matrix keratinocytes.

Abnormalities of human skin pigmentation can occur as a result of both genetic and environmental factors. Exposure of the skin (especially Caucasian) to ultraviolet radiation, particularly in the UVB (i.e. intermediate) wavelengths, upregulates synthesis of melanocyte tyrosinase resulting in increased melanogenesis and thus tanning. However, acute or persistent UVB exposure can result in the formation of hyperpigmented lesions or regions of skin, including malignant melanoma skin cancer. Both actinic damage and constitutional abnormalities can produce affected regions such as melasma, age spots, liver spots, freckles and other lentigenes.

Vitiligo is the converse of hyperpigmentation, in which cutaneous melanocytes are either ablated or fail to produce sufficient pigment. Although it would be desirable to restore lost pigmentation in vitiligo-affected skin with topical therapies, this has proven to be quite difficult to accomplish in a high proportion of subjects. As an alternative to pigmentation therapy or cosmetic camouflage with dihydroxyacetone sunless-tanning lotions, one might reduce the normal pigmentation of the unaffected skin to reduce contrast.

Some purportedly “active” or “functional” agents for lightening skin color (e.g., arbutin, kojic acid, niacinamide, licorice, magnesium ascorbyl phosphate, among others) have not been demonstrated yet to be clinically efficacious. The U.S. FDA-approved pharmaceutical products containing 2-4% hydroquinone (“HQ”) are minimally to moderately efficacious. However, HQ has been demonstrated to be cytotoxic to cultured mammalian melanocytes, and mutagenic in Salmonella and mammalian Chinese hamster V79 cells. Hydroquinone's in vitro mechanism of action appears to be primarily a melanocytic cytotoxic effect.

Skin Texture/Wrinkling and Acetyl-Coenzyme A (CoA) Carboxylase or Fatty Acid Synthetase

The skin is the largest organ of the body and protects the body from the environmental damage. This protection is provided by the stratum corneum or horny layer of the skin. In this regard, the stratum corneum acts as a barrier (also known as “water barrier” or “permeability barrier”) between the body and the outside environment.

The stratum corneum lipids are the key constituents for a functional barrier. Major classes of stratum comeum lipids include cholesterol, free fatty acids, and ceramides. These lipids are synthesized inside the epidermal cells of the skin and are then secreted into the space between these cells, where they assemble into lamellar bilayer sheets to provide a permeability barrier. The stratum corneum serves as a gate keeper that prevents the entry of infection, chemicals, and other pollutants into the skin. In addition, the stratum corneum prevents the loss of moisture from the skin and thus helps maintain a proper intracellular milieu for normal cellular functions.

In addition to providing a permeability barrier, skin lipids are important for the maintenance of the skin's shape, form, and healthy youthful appearance. Therefore, the skin lipid, its integrity, amount, and the ability to renew itself are crucial for esthetic appearance, such as decreasing wrinkles and other signs of aging. During youth, the blood circulation delivers to the skin all the necessary ingredients for lipid synthesis. However, as we age, the blood flow to the skin decreases, which results in decreased delivery of the lipid building nutrients to the skin. The net result is diminished lipid synthesis and decreased lipid contents of the skin of the aging population.

Depletion and inadequate replenishment of skin lipids leads to moisture loss, dryness, skin wrinkles, and altered appearance. Therefore, restoration of skin's lipid contents is crucial for both health and esthetic reasons. To improve the skin barrier, publications disclose compositions containing natural or synthetic skin lipids. For example, U.S. Pat. No. 5,643,899 discloses the use of lipids for epidermal moisturization and repair of barrier function. However, it is uncertain whether the lipid composition of these products mimic the composition of the human skin lipids. These products contain only from one to three types of lipids, whereas skin lipids are made up of hundreds of types of lipids.

Lipids in skin care products may have been derived from human and/or animal tissues and thus carry the risk of being contaminated with microorganisms such as viruses and/or bacteria. Furthermore, because lipids in general are unstable, the lipids in these products may undergo peroxidation, and the peroxidation products of lipids may cause harm to the skin. Finally, some exogenous lipids, including ceramides, can actually impede rather than improve the skin's barrier functions. Because of these limitations and concerns about these products, cosmetic compositions which can enhance endogenous production of a correct mix of a full spectrum of physiological lipids by the epidermal cells are highly desirable.

The role of branched-chain acyl coenzyme A (CoA) to produce fatty acids has been known for years (Nicolaides: Science, 186: 19-26, 1974). Recently, carbon skeletons of branched-chain amino acids has been incorporated into skin lipids in animals (Oku et. al.: Biochim. Biophys. Acta 1214: 279-287, 1994). U.S. Pat. No. 5,472,698 discloses a composition containing lipid building ingredients (serine or its derivatives). However, these ingredients are capable of producing a single class of skin lipids, namely ceramides, and do not include components to produce a full spectrum of skin lipids, namely cholesterol, free fatty acids, and ceramides.

Acetyl-CoA carboxylase and fatty acid synthetase are the two major enzymes involved in the synthesis of fatty acids in animals. The activities of both enzymes are affected by nutritional manipulations. Although acetyl-CoA carboxylase is considered generally to be the rate-limiting step in lipogenesis, there is evidence that fatty acid synthetase may become rate limiting under certain conditions.

The principal support for the view that acetyl-CoA carboxylase is the rate-limiting enzyme for lipogenesis is that the activity of the enzyme is controlled by allosteric effectors that change the catalytic efficiency of the enzyme. Fatty acid synthetase is subject to the type of control necessary for an enzyme to serve as a regulator of the rate of a biological process over a short term.

Skin/Hair Color and Tyrosinase

Tyrosinase is the key enzyme for melanin biosynthesis. Disorders of tyrosinase activity include Parkinson's disease, vitellego and albinism. Tyrosinase is a ubiquitously distributed copper-containing monoxygenase that is essential for melanin biosynthesis in pigment cells. It catalyzes the conversion of tyrosine to dihydroxyphenylalanine (DOPA) and the conversion of DOPA to dopaquinone, referred to as tyrosine hydroxylase activity and DOPA oxidase activity, respectively.

Disorders of tyrosinase expression and melanin biosynthesis are related to many diseases involving pigmentation such as albinism, hair pigment loss, and vitellego. Tyrosinase is a key enzyme for melanin synthesis in vertebrate pigment cells, melanocytes, and retinal pigment epithelial cells. Tyrosinase is absent in human white hair bulbs, as well as in albino epithelial cells. Thus, the loss of tyrosinase could be the basis of pigment loss in hair.

Skin Condition and Superoxide Dismutase

Active oxygen liberated in a living body must be rapidly consumed. Otherwise, various cell elements such as DNA, lipids and proteins become the target molecules for oxidation, and breakdown of the functions of the cells accompanies the production of lipid peroxides.

Superoxide dismutase (SOD) has is known as a catalyst for decomposing and detoxifying superoxides. When SOD is applied externally to the skin, SOD lowers the amount of lipid peroxides (LPO) in the epidermis due to ultraviolet rays. (R. Ogura et. al., The Biological Role of Reactive Oxygen Species in Skin, edited by O. Hayaishi, S. Imamura and Y. Miyachi, University of Tokyo Press, 1987, p. 55).

Intravenously injected SOD derivatives prevent or considerably alleviate cerebral ischemic disorders, myocardial ischemic disorders, acute gastric mucosal disorders, carrageenin edema, hemorrhagic shock, cerebral edema, renal ischemic disorders, etc. (M. Inoue and N. Watanabe: “Antioxidants in Therapy and Preventive Medicine,” edited by I. Emerit, Plenum Press, 1990).

The present invention is directed to compositions and methods for treating skin conditions with a fusion protein having SOD activity. Examples of the treatable skin conditions include, but are not limited to, wrinkles, skin pigmentation, sun burn, and other types of skin bums. In one embodiment, the fusion protein comprises a peptide having SOD activity and a signal peptide or membrane transport sequence. In a preferred embodiment, the fusion protein contains a membrane transport sequence from HIV Tat protein.

Signal Peptides

Signal peptide sequences guide the translocation of most intracellular secretory proteins across the endoplasmic reticulum (ER) and plasma membranes through protein-conducting channels. Secretory protein transport also support a role for the signal sequence in targeting proteins to certain cellular membranes (B. Alberts et. al., Molecular Biology of the Cell, Third Edition, Garland Publishing (1994) pp. 557-585).

Several types of signal sequence-mediated translocation pathways from have been proposed for exiting from the interior of the membrane. The major model implies that the proteins are transported across membranes through a hydrophilic protein-conducting channel formed by a number of membrane proteins.

In eukaryotic cells, newly synthesized proteins in the cytoplasm are targeted to the ER membrane by signal sequences that are recognized generally by the signal recognition particle (SRP) and its ER membrane receptors. This targeting step is followed by the actual transfer of protein across the ER membrane and out of the cell through the protein-conducting channel. In bacteria, the transport of most proteins across the cytoplasmic membrane also requires a similar protein-conducting channel. On the other hand, signal peptides can interact strongly with lipids, so transport of some secretory proteins across cellular membranes can occur directly through the lipid bilayer in the absence of any proteinaceous channels.

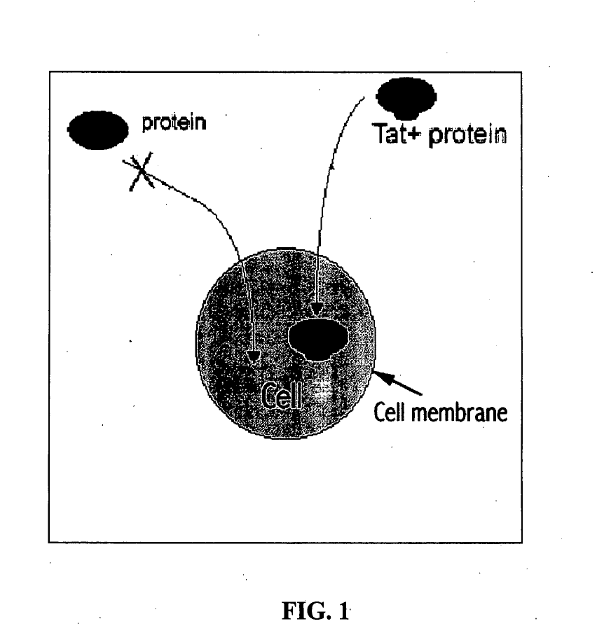

Another aspect of the present invention provides a method for importing a biologically active molecule into a cell using mechanisms naturally occurring in cells and thus avoiding damaging the target cells. As shown in FIG. 1, attachment of a membrane transport sequence, such as the membrane transport sequence of HIV Tat protein, to a protein would facilitate the entry of the protein into a cell. Additionally, the present method can be used to import molecules into large numbers of cells upon topical application to the skin exterior and employed in the treatment of numerous skin and hair conditions.

Localization Signal Peptides for Fusion Proteins in Active Agent Delivery

Importing exogenous biologically active proteins into the cells of the epidermis or dermis can be accomplished by forming a fusion having an importation competent signal peptide sequence fused to a selected biologically active enzyme or enzyme inhibitor protein and administering the fusion protein to the cell by topical application onto the skin. The complex is then imported across the cell membrane by the cell. Thus, one embodiment of the present invention is to provide a method of importing a fusion protein into an epidermal cell by topical application of the active agent fusion protein within a cream, ointment or tonic.

Specific targeting of tissues or cells with peptides depends on the presence of unique or differentially expressed markers on cells. The plasma membrane of eukaryotic cells is the first barrier which must be traversed by agents acting on intracellular targets. In the detailed description that follows, certain specific sequences have been identified according to the invention that can expedite transport when fused to the active agent enzymes and inhibitors identified above.

The intracellular action of the enzyme or enzyme inhibitor is known to play a critical role in regulating cellular activity in affecting, for example, skin and hair conditions. The present invention succeeds in regulating intracellular activity by delivery of the fusion protein through topical application and then inducing the desired cellular response in a mammal, to improve skin and hair conditions.

Short cellular sequences capable of directing the movement of a “cargo” enzyme or protein have now been identified. These sequences function either via endocytic pathways or through a proposed mechanism referred to as ‘inverted micelles.’ Based upon their amino acid sequence, all known import signals can be broadly classified as either hydrophobic, amphipathic or cationic.

The specific import signals utilized according to the present invention are shown in table 1:

TABLE 1

| | | |
|---|---|---|
| IMPORT | | |
| SIGNAL | SOURCE | AMINO ACID SEQUENCE |

Hydrophobic sequences

| | | |
|---|---|---|
| Membrane | Karposi FGF | AAVALLPAVLLALLAP |
| Permeable | | (SEQ ID NO:1) |
| Sequences(MPSs) | | |
| | Grb2 (SH2 domain) | AAVLLPVLLAAP |
| | | (SEQ ID NO:2) |
| | Integrin β3 | VTVLALGALAGVGVG |
| | | (SEQ ID NO:3) |
| Fusion sequence | HIV-1 GP41(1-23) | GALFLGFLGAAGSTMGA |
| | | (SEQ ID NO:4) |
| Signal sequence | Caiiman croc | MGLGLHLLVLAAALQGAMGLGLHLLLAAALQGA |
| | Lg(v) light chain. | (SEQ ID NO:5) |

Amphipathic/Cationic Sequences

| | | |
|---|---|---|
| KALA | Influenza HA-2 | WEAKLAKALAKALAKHLAKALAKALKACEA |
| | (1-20) | (SEQ ID NO:6) |
| GALA | | WEAALAEALAEALAEHLAEALAEALEALAA |
| | | (SEQ ID NO:7) |
| 4₆ | | LARLLARLLARLLRALLRALLRAL |
| | | (SEQ ID NO:8) |
| HEL 11-7 | | KLLKLLLKLWKLLLKLLK |
| | | (SEQ ID NO:9) |
| Penetratin or | Antennapedia | RQIKIWFQRRMKKWK |
| Antp | third helix | (SEQ ID NO:10) |
| | (43-58) | |
| Tat | HIV-1 Tat | YGRKKRRQRRR |
| | (47-57) | (SEQ ID NO:11) |
| VP22 | HSV transcription | DAATATRGRSAASRPTERPRAPARSASRPRRPVE |
| | factor (267-300) | (SEQ ID NO:12) |
| Transportan | Galanin + | GWTLNSAGYLLGK1NLKALAALAKKIL |
| | Mastoparan | (SEQ ID NO:13) |

One group of hydrophobic sequences called membrane permeable sequences (MPSs) is derived from the hydrophobic region of various signal sequences. MPSs adopt a characteristic α-helical conformation under membrane mimetic environments, despite the lack of primary sequence homology between the signal sequences. These hydrophobic regions can be from about 18 to 21 amino acids long. They traverse the cell membrane and are therefore able to import covalently attached functional domains from other intracellular proteins. Examples of such domains include the src homology 2 (SH2) domain of Grb2, human integrin proteins β1, β3 and α_{lib} and the Nuclear Localization Signal (NLS) of NFkB p50. Other hydrophobic signal sequences [HIV gp4l fusion peptide, Caiiman crocodylus immunoglobulin (v) light chain signal sequence] have also been fused to the NLS sequence derived from the SV40 large T antigen to target the nucleus of cells and deliver antisense oligonucleotides and plasmid DNA.

Amphipathic sequences harbor a periodicity of hydrophobic and polar residues. These sequences, typified by the fusion peptide of influenza hemagluttinin (HA-2) and related synthetic analogs [GALA, KALA, 4₆ and Hel 11-7] represent a group of import signals that have been shown to interact with cellular membranes. Their interaction with the uncharged lipid bilayers results in fusion events with the membrane. The lower pH present in vesicles causes these sequences to undergo a random coil to α-helical transition that induces leakage of vesicular contents. The peptides 4₆ and Hel 11-7 have been shown to transport plasmid DNA into adherent cell lines.

Cationic peptide sequences represent the final group of import signals. Polylysine sequences have been used for several decades as a method of importing various macromolecules across the cell membrane. These sequences interact with the negatively charged phospholipids of the cell membrane and enter the cell via the endocytic pathway. Penetratin from the third helix of the Antp and Transportan created from the fusion of galanin to mastoparan sequences, penetrate cell membranes via a postulated inverted micelle pathway.

These signal sequences, when coupled to an enzyme or protein cargo sequence form fusion proteins for transport into the intracellular regions of the skin cells that then modulate the enzymatic pathways associated with hair and skin conditions.

Formulations, Dosage and Administration

The present fusion proteins may be formulated in compositions for delivery via an appropriate route using formulations known in the art for other topical applications, for instance, as described in various U. S. patents cited herein. Those skilled in the art will appreciate that the disclosed compositions of the present invention are aqueous or non-aqueous preparations for administration to mammals, and preferably humans.

The present invention is further illustrated by the following examples which should not be construed as limiting. The contents of all references, patents and published patent applications cited throughout this application, as well as the Figures and Tables are incorporated herein by reference.

### EXAMPLE 1

### Construction and Expression of Tat-GFP Fusion Protein

Preparation of the Fusion Protein

GFP (Green Fluorescent Protein) is a marker for illustrating the distribution of a protein composition in a cell population sample and can demonstrate intracellular delivery across the cell membrane in a fusion protein according to the invention. A Tat-GFP expression vector (gift from Molecular medicine laboratory, International center for genetic engineering and biotechnology, Trieste, Italy) was transformed into E coli BL21. Clones with the expected insert was selected using BamH I and EcoR I restriction analysis, confirmed by DNA sequencing, and grown in TB broth (12 g/L Tryptone, 24 g/L Yeast Extract, 4 g/L Glycerol, 2.1 g/L KH₂PO₄, 14.7 g/L K₂HPO₄) overnight with 100 mg/L ampicillin at 30° C. The 10% inoculation was cultured at 25° C. with vigorous shaking. Protein expression was induced with 0.5 mmol/L IPTG when OD₆₀₀ was around 1.0. After induction for 5 hours, cells were harvested and sonicated for 15 min in sonication buffer (50 mmol/L Tris-HCl, pH 8.0, 50g/L glycerol, 150 mmol/L NaCl,). Recombinant Tat-GFP was prepared from the cell lysate.

### EXAMPLE 2

### Delivery of Tat-GFP Fusion Protein in Culture Cells and C. elegans

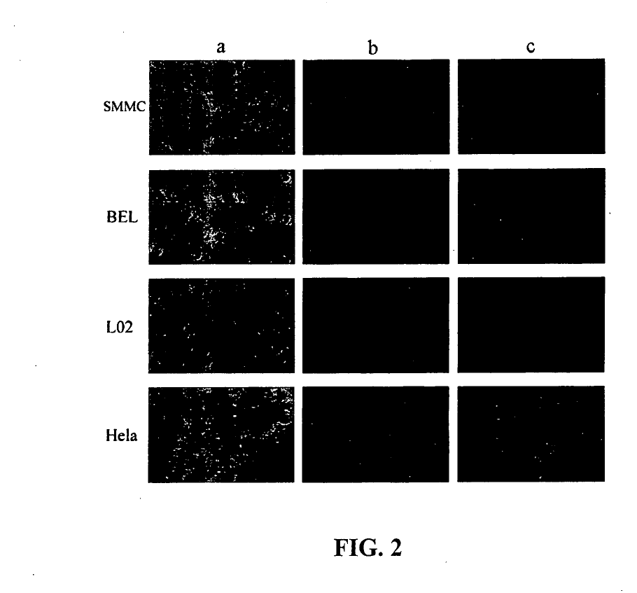

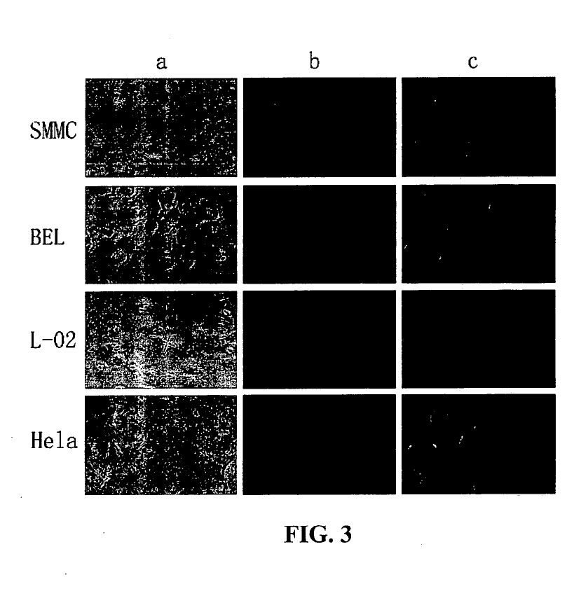

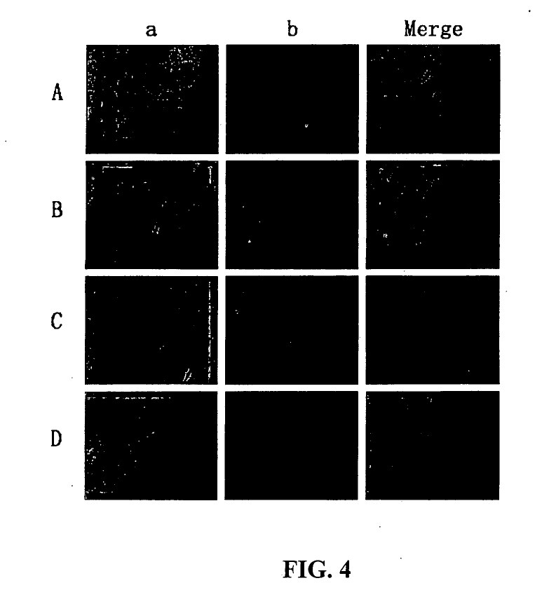

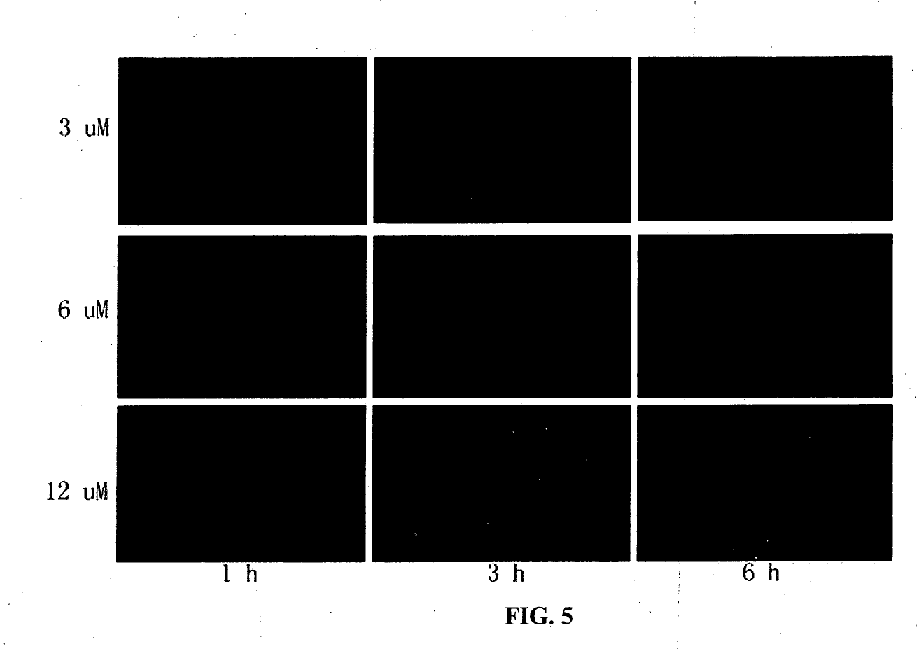

Human hepatic carcinoma cells (SMMC-7721 and BEL-7402), human normal hepatic cells (L02) and human cervical carcinoma cells (Hela) were seeded in DMEM or RPMI1640 containing 10% fetal bovine serum (FBS) and incubated at 37° C. with 5% CO₂ fro 24 hours. Tat-GFP was then added to the culture to a final concentration of 4 umol/L for 6 h. The Tat-GFP fusion protein was efficiently internalized into SMMC-7721, BEL-7402, L02 and Hela cells (FIG. 2). In contrast, control GFP protein was not internalized in these cell lines ( FIG. 3). As shown in FIG. 4, the internalization of Tat-GFP in cultured cells is concentration-dependent. Similar results were also obtained in C. elegans (FIG. 5).

### EXAMPLE 3

### Topical Application of Tat-GFP Fusion Protein

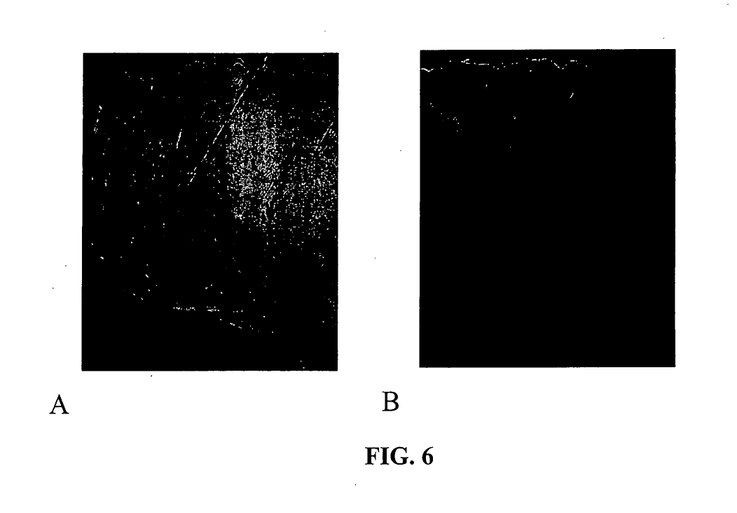

Tat-GFP fusion protein was tested on mouse skin. Briefly, Tat-GFP fusion protein in PBS (2 mg/kg) was topically applied onto the skin of mice. After 1 hour, the mice were sacrificed. The skin was harvested and embedded in OCT. Thin frozen sections (7 μm) were cut on a cryomicrotome, fixed in 4% (v/v) formaldehyde, washed three times, and examined with fluorescence microscopy. As shown in FIGS. 6, the Tat-GFP fusion protein was delivered to the hair follicles of the mouse skin.

### EXAMPLE 4

### Expression of Tat-SOD Fusion Protein

Tat-SOD Fusion Protein was expressed in E. coli BL21(DE3) under the induction of IPTG from recombinant vector pGEX-Tat-SOD. Recombinant fusion protein Tat-SOD was purified by affinity chromatography and enzymatic digest.

Thermal stability: the Tat-SOD fusion protein was incubated at 30° C., 40° C., 50° C., 60° C., 70° C. or 75° C. for 60 minutes and than assayed SOD activity. It was found that the enzymatic activity was stable under 40° C. but decreased by 20% and 25 % at 60° C. and 70° C., respectively.

pH stability: the Tat-SOD was incubated for 90 minutes with buffer of different pH. It was found the Tat-SOD fusion protein was stable between pH 5.5-11, which is the pH value of most biological product.

### EXAMPLE 5

### Internalization of Tat-SOD Fusion Protein in Cultured Cells

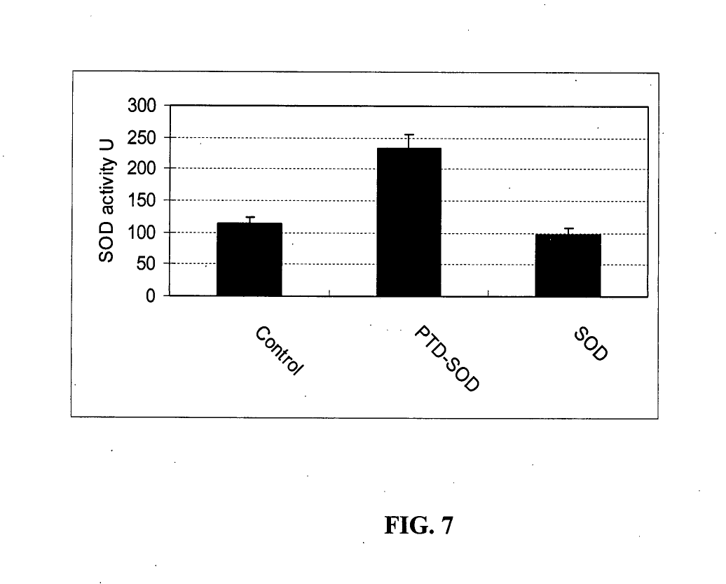

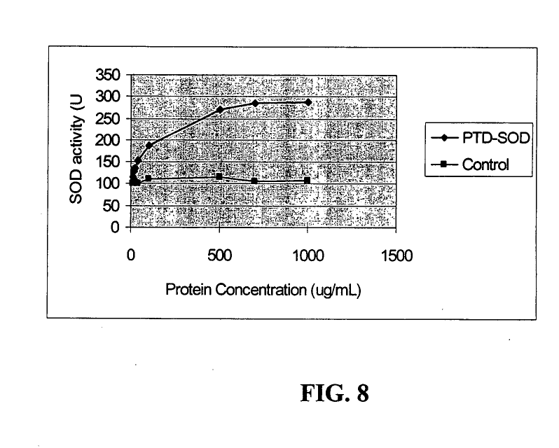

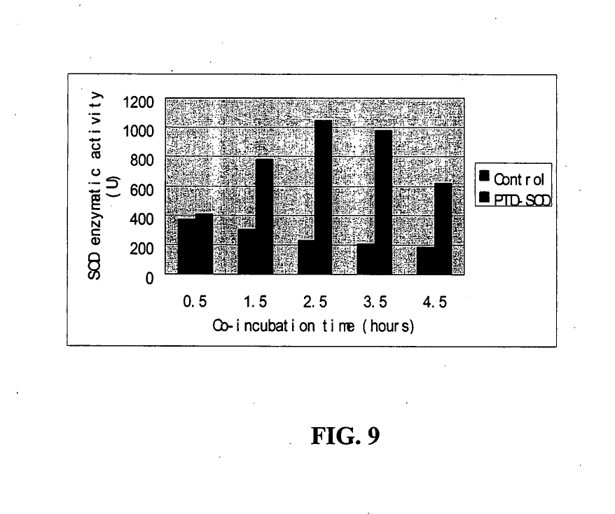

Hela cell co-cultured with 500 μg/mL SOD or Tat-SOD in fresh RPMI 1640 medium PBS buffer was used as control). After 4 hours , the total SOD activity of cell lysate was assayed spectrophotometrically at 505 nm with a commercial kit of xanthine-xanthine oxidase system (SOD assay kit, Cat. No. A001; Jian Cheng Institute of Biotechnology, Nanjing, China). As shown in FIG. 7, Tat-SOD was effectively internalized in Hela cells. The internalization of Tat-SOD is concentration-dependent (FIG. 8). In another experiment, Mitochondria from liver cells of rat were incubated with 500 μg/mL Tat-SOD in fresh RPMI 1640 medium (PBS buffer was used as control) for 0.5, 1.5, 2.5, 3.5, or 4.5 hours . Trypsin was then added to remove the Tat-SOD fusion proteins attached on the mitochondria surface. The mitochondria was lysed and the total SOD activity of mitochondria lysate was assayed spectrophotometrically at 505 nm with a commercial kit of xanthine-xanthine oxidase system (SOD assay kit, Cat. No. A001; Jian Cheng Institute of Biotechnology, Nanjing, China). As shown in FIG. 9, internalization of Tat-SOD in mitochondria peaks at 2.5 hours.

### EXAMPLE 6

### Effect of Tat-SOD Fusion Protein on Damaged Cells

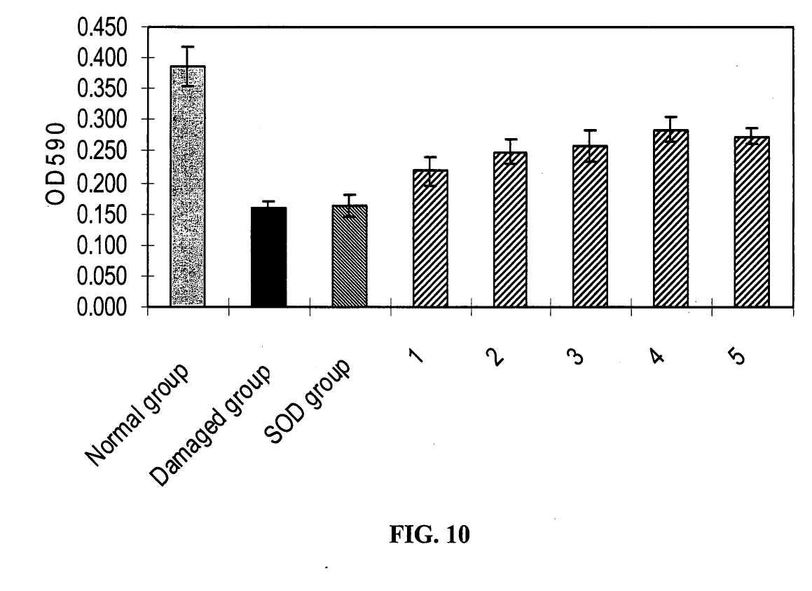

MDCK cells(Madin-Darby canine kidney cell line) were inoculated to 96-well culture plate and incubated overnight. Cells in the normal group were not treated with Alloxan. Cells in the damaged group were treated with 10 umol/L Alloxan for an hour, washed, and incubated with culture media for 24 hour. Cells in the SOD group were treated with 10 umol/L Alloxan for an hour, washed, and incubated with culture media containing 6 mg/ml SOD for 24 hour. Cells in groups 1-5 were treated with 10umol/L Alloxan for an hour, washed, and incubated with culture media containing Tat-SOD at 0.05 , 0.1 , 0.2 , 0.4 and 0.6 mg/ml, respectively, for 24 hour. Cell damage was determined with MTT method at 590 nm. Compared to control cells, Tat-SOD treated cells showed improved recovery from Alloxan damage (FIG. 10).

### EXAMPLE 7

### In Vivo Delivery of Tat-SOD Fusion Protein

(A) Intraperitoneal (i.p.) delivery of Tat-SOD

Electrophoretically pure Tat-SOD was dissolved in PBS buffer and administered intraperitoneally into mice, in a dosage of 2000 U/kg body weight. Tissue samples from heart, liver and brain of the mice were prepared 4 hours thereafter. Each sample was homogenized in 10 folds of physiological saline, and was then, centrifuged for 30 minutes at 15,000 g and 4° C. The supernatant was aspirated and the total SOD activity was assayed spectrophotometrically at 505 nm with a commercial kit of xanthine-xanthine oxidase system (Cat. No. A001; Jian Cheng Institute of Biotechnology, Nanjing, China). PBS buffer was used as control. As shown in Table 2, significant increases in SOD activity were found in liver and brain. Data was processed by STDEV function and expressed as means±SD of 10 animals for each group. The difference between the means of two groups was evaluated with a test and considered significant at P<0.05

TABLE 2
Tissue SOD activity after i.p. injection of Tat-SOD

| | Heart | Liver | Brain |
|---|---|---|---|
| PBS control | 4.00 ± 0.16 | 11.02 ± 14.37 | 2.18 ± 0.46 |
| S-SOD group | 4.13 ± 0.24 | 14.23 ± 12.24 | 2.62 ± 0.58 |
| Increment (%) | +3.2 | +29.1 | +20.1 |
| P value | 0.12 | 0.05 | 0.001 |

(B) Oral Delivery of Tat-SOD

After acclimating the environment for one week, 38 mice were divided randomly into 4 BW-matched groups. The mice of control group were sacrificed before the administration of Tat-SOD. Groups 1, 2 and 3 were sacrificed after 2, 4 and 6 hours of oral administration of 500 U enzymatic activity of Tat-SOD in PBS buffer, respectively. Brain, heart and liver were put into pre-cold physiological saline to wash blood attaching on the surface of tissues; subsequently redundant solution on the surface of tissues was removed by the adsorption of filter papers, and the connective part were removed carefully. Then the tissue was homogenized in 10 folds of pre-cold physiological saline by glass homogenizer. The homogenate was centrifuged (3000 rpm, 15 minutes) and the supernatant was prepared for the spectrophotometrically assay of enzymatic activity of SOD at 505 nm with a commercial kit of xanthine-xanthine oxidase system (Cat. No. A001; Jian Cheng Institute of Biotechnology, Nanjing, China). Data was processed by STDEV function and expressed as means±SD for animals in each group. The difference between the means of two groups was evaluated with a TTEST and considered significant at P<0.05. As shown in Table 3, there was a significant increase of SOD activity in liver and brain after oral delivery of Tat-SOD.

TABLE 3
Tissue SOD activity after oral delivery of Tat-SOD

| | Animal | SOD activity (U/mg tissue ) | | |
|---|---|---|---|---|
| Group | number | Heart | Liver | Brain |
| Control (0 h) | 10 | 4.30 ± 0.23 | 12.27 ± 0.46 | 1.33 ± 0.14 |
| Group 1 (2 h) | 8 | 4.70 ± 0.27 | 15.89 ± 1.45** | 1.33 ± 0.15** |
| Group 2 (4 h) | 10 | 4.76 ± 0.40 | 16.00 ± 0.72** | 1.70 ± 0.12** |
| Group 3 (6 h) | 10 | 4.76 ± 0.31 | 14.02 ± 0.57** | 1.57 ± 0.19** |

**P < 0.01 vs control

### EXAMPLE 8

### Formulation of Tat-SOD Fusion Protein

A Tat-SOD cream was formulated for topical application. The cream contains 200 u SOD activity/ml vehicle. The vehicle was composed of water (20-80%), glyceryl stearate (0.25-12%), cetyl alcohol(0.1-1 1%), propylene glycol stearate (0.1-11%), polysorbate 60 (0.1-5%), sorbitan stearate (0.05-5%), Vitamin E (0.02-4%), methylparaben (0.01-4%), propylparaben (0.01-4%), and butylated hydroxyanisole (BHA) (0.01-4%).

### EXAMPLE 9

### Wrinkles Removal with Tat-SOD Fusion Protein

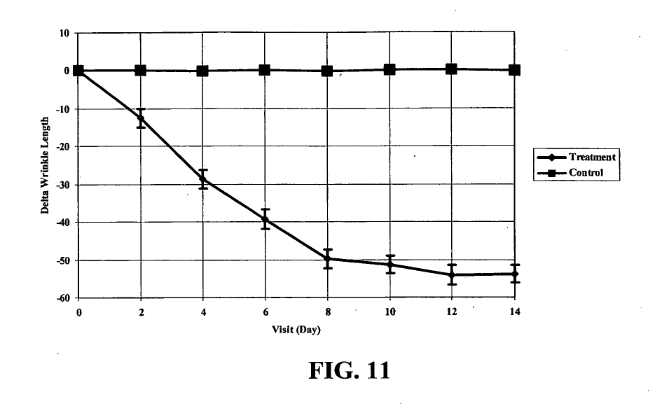

Subjects with Fitzpatrick skin Type III or Type IV were recruited for this clinical trial. There were no pregnant females or breast feeding mothers involved in this study, and there were no concomitant use of estrogen or oral contraceptives by the subjects. None of the subjects had any allergy to SOD or any other known allergic history. None of the subjects had active herpes simplex infection or a history of hypertrophic scars or keloids. Thirty subjects were involved in the trial. The age distribution among the subjects were: 20's (12), 30's (6), 40's (6), 50's (4), 60's (2). The subjects were equally divided into two groups by random choices: (1) the treatment group, in which applications of Tat-SOD cream at 200 units/mL were performed 3 times a day for 2-weeks, and (2) the control group, in which applications of a non-Tat-SOD cream (vehicle only) were performed 3 times a day for 2-weeks. The study was performed at Fujian Union Hospital, Dermatological Department. As shown in FIG. 11, the Tat-SOD cream significantly reduced wrinkle length.

### EXAMPLE 10

### Skin Whitening with Tat-SOD Fusion Protein

Twenty healthy men in their 20's or 30's were tested. After obtaining informed consents from the volunteers, UVB-induced hyperpigmentation was elicited on the inside skin of the upper arm. Four separate areas (1.2 cm×1.2 cm) on the inside skin of each upper arm were exposed to UVB radiation (SUV-100 UVB Lights, UVB radiometer, Shanghai Sigma Hightech Co., Ltd., China) from 5 to 7 times a week for two consecutive weeks until substantial hyperpigmentation was achieved in each person. The UVB intensity was 1 mW/cm², and the total energy dose was 1.2-fold a minimal erythema dose (MED) per day for each person. Tat-SOD cream (200units/mL) application was started 2 days after the final UVB radiation, and was then topically applied daily to the hyperpigmented areas (0.2 ml/cm²) 2 times every day for 2 successive weeks. The degree of pigmentation was assessed as the L-value measured with a chromameter (CR-100, Minolta, Japan) once every two days from the beginning of Tat-SOD cream application.

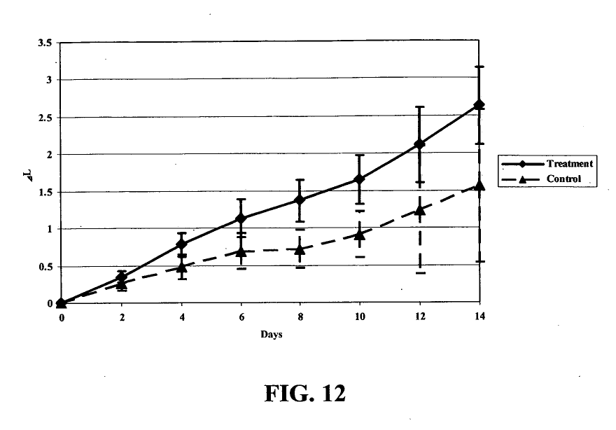

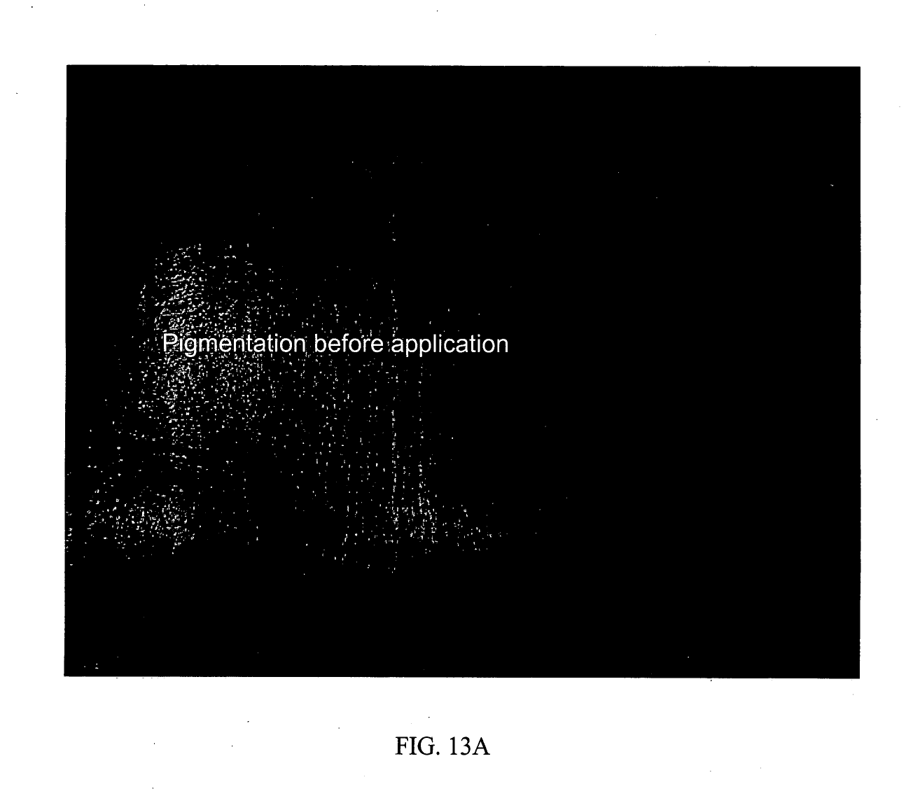

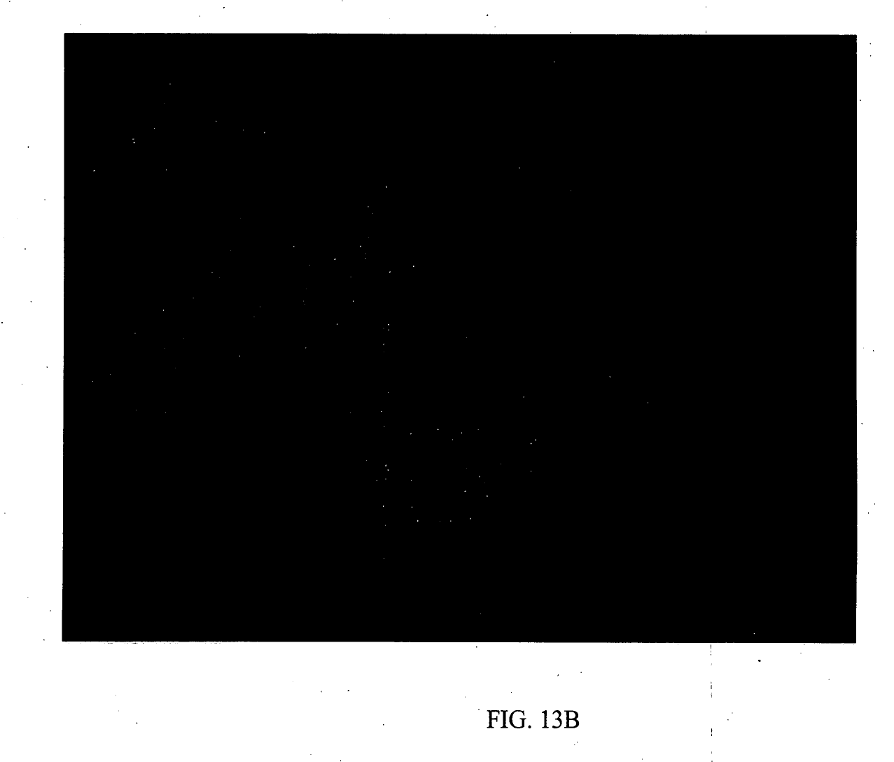

Depigmentation with Tat-SOD cream on the brachium was shown in FIG. 12. The Tat-SOD cream demonstrated a remarkable decrease in hyperpigmentation compared with the non-treatment control. FIGS. 13A and 13B show hyperpigment before and after the treatment, respectively.

### EXAMPLE 11

### Sunburn Treatment with Tat-SOD Fusion Protein

UVB induced skin erythema was monitored by means of a reflectance visible spectrophotometer X-Rite mod.968, having 0° illumination and 45° viewing angle. The instrument was calibrated with a supplied white standard traceable to the national standard's perfect white diffuser. Reflectance spectra were obtained over the wavelength range 400-700 nm using illuminant C and 2° standard observer.

In vivo experiments were performed on 10 healthy volunteers (both sexes) of skin types II and III, with a mean age of 29±7 years. All the volunteers were fully informed of the nature of the study and the procedures involved and gave their written consent. The subjects did not suffer from any ailment, were not on medication at the time of the study and were rested for 30 minutes prior to the experiments. Room conditions were set at 25±2° C. and 40-50% relative humidity.

Skin erythema was induced by UVB irradiation using an ultraviolet lamp (SUV-100 UVB Lights, UVB radiometer, Shanghai Sigma Hightech Co., Ltd., China), which emitted in the range 290-320 nm. The flux rate measured at the skin surface was 0.80 mW cm⁻². For each subject, the minimal erythema dose (MED) was determined preliminarily and an irradiation dose corresponding to the double of the MED was used throughout the study.

For each subject, six sites on the ventral surface of one upper arm were defined using a circular template (1 cm²) and demarcated with permanent ink. Freshly prepared creams with or without Tat-SOD was employed as treating formulation and control. Skin sites were exposed to UVB irradiation and then 0.1 ml of creams were immediately applied to each of the irradiated sites for one hour using a chamber. For each subject, two skin sites were treated by non-Tat-SOD cream, but exposed to UVB radiation (control).

After the treatment period, the chambers were removed, the skin surfaces were gently washed with wet tampon; after which the UVB-induced erythema was monitored for 36 h using the reflectance spectrophotometer described following.

From the skin spectral determination, the erythema index (EI) was calculated using the following equation (Dawson et al., Phys. Med. Biol. 1980, 25 695-709)

$$EI = 100 [ log \frac{1}{{R}_{560}} + 1.5 ( log \frac{1}{{R}_{540}} + log \frac{1}{{R}_{580}} ) - 2 ( log \frac{1}{{R}_{510}} + log \frac{1}{{R}_{610}} ) ]$$

where 1/R is the inverse reflectance at a specific wavelength (560, 540, 580, 510, 610 nm). EI baseline values were taken at each designated site before UVB irradiation and were subtracted from the E1 values obtained at each time point, to determine ΔEI values following UVB exposure. For each site, the area under the response ΔEI/time curve (AUC) was computed using the trapezoidal rule.

AUC values were inversely related to the ability of the formulations tested to inhibit UVB skin erythema. To better compare the efficacy of the different products tested the percentage inhibition of UVB skin erythema (PIE) was calculated from AUC values using the following equation:

$$Inhibition \% ( PIE ) = \frac{{AUC}_{( C )} - {AUC}_{( T )}}{{AUC}_{( C )}} \times 100$$

where AUC_{(C)} is the area under the response time curve of sites which received no treatment (control), AUC_{(T)} is the area under the response time curve of the sites treated with the solutions being tested. Statistical analysis was performed by using Student's t-test.

Table 4 AUCO₀₋₇₂ values obtained, in healthy volunteers, treating with Tat-SOD control and placebo, after skin exposure to UVB radiation.

TABLE 4

| | AUC₀₋₇₂ | PIE |
|---|---|---|
| Tat-SOD cream Treatment | 1053.6 ± 76.5 | 42.41 |
| Non-Tat-SOD cream Control | 1829.4 ± 123.6 | |

P < 0.01, n = 5

To assess the protective effect of Tat-SOD cream against UVB-induced erythema, the extent of erythema in human volunteers was monitored by means of reflectance spectrophotometry. Because skin erythema is due to increased hemoglobin content in skin vessels, El values are calculated by subtracting LIR values at 510 and 610 nm (mainly due to melanin absorption) from the sum of hemoglobin LIR values at 540, 560 and 580 nm, which represent the wavelengths of hemoglobin absorption peak (Dawson et al., supra).

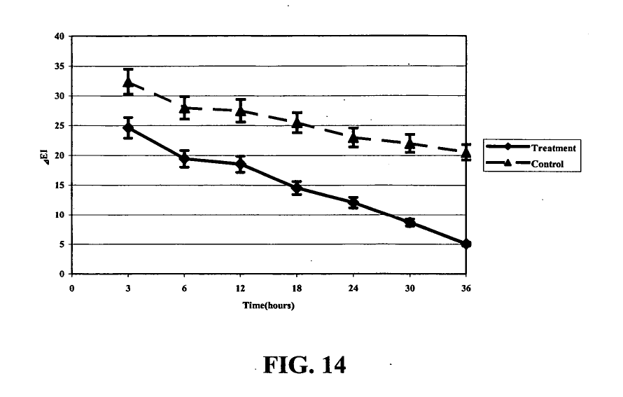

The time course of erythema for skin sites treated with cream of Tat-SOD and placebo after UVB irradiation is shown in FIG. 14. From ΔEI versus time plots, the area under the response time curve (AUC₀₋₇₂) was computed using the trapezoidal rule. AUC₀₋₇₂ values are reported in Table 4.

As shown in FIG. 14, Tat-SOD cream provided a significant protection to the skin against UVB-induced erythema, which illuminated the protective effects of Tat-SOD on the phospholipidic biomembranes against UV light-induced peroxidation. Tat-SOD cream's PIE value was 42.41%.

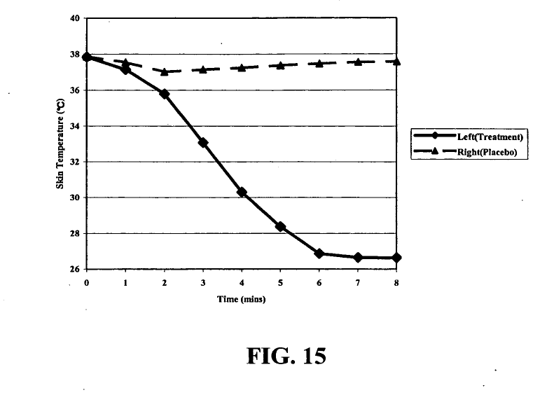

In another experiment, naked arms were exposed to sunshine for a whole day and developed erythema. In the next day, the left arm was applied with TAT-SOD cream with a SOD activity of 1000 U/mL, and right was kept un-treated. The temperature on the surface of both arms was measured for 8 minutes. As shown in FIG. 15 , Tat-SOD cream also reduces skin temperature in sun burn area within minutes of application.

### EXAMPLE 12

### Skin Burn Treatment with Tat-SOD Fusion Protein

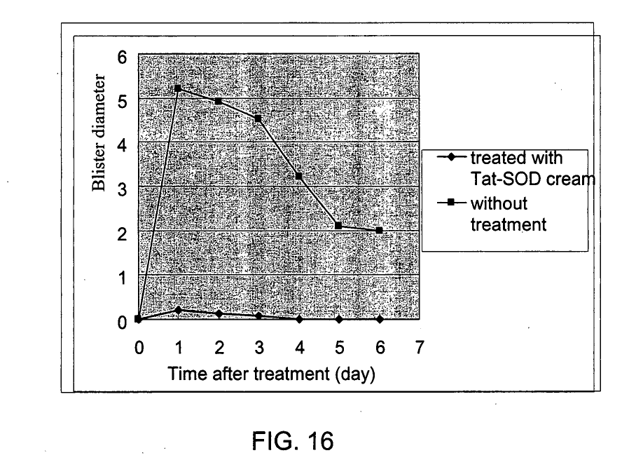

Human skin was spattered by boiling cooking oil (arawana brand, made by Southseas oil & fat industrial Limited, Chiwan, China). The scald was treated with immediately with TAT-SOD cream with a SOD activity of 1000 U/mL. The blister diameter developed in the spattered zone was measured for 6 days. As shown in FIG. 16, Tat-SOD cream stopped the development of blisters after scalding by boiling oil. The unit for blister diameter is in mm.

### EXAMPLE 13

### Tat-Tyrosinase Fusion Protein Preparation and Method of Enhancing Pigmentation of Hair and Skin

A Tat-Tyrosinase fusion protein can be produced by chemical synthesis or by recombinant method. The fusion protein can have the Tat signal sequence covalently attached at the amino-terminal region, the carboxy-terminal region or at any other region of the enzyme, so long as the covalently attached Tat signal sequence which will not significantly interfere with the activity of the Tyrosinase enzyme.

A fusion protein thus prepared can be formulated in a cream or ointment for topical application to the skin to enhance pigmentation of the skin or hair in cells proximate to the application area. In another embodiment, a Tat-Tyrosinase fusion protein of this invention is applied or administered to the skin during an appropriate period and using a sufficient number of dosages to achieve enhanced skin pigmentation. The concentration of active agent in the composition will depend on absorption, inactivation, and excretion rates of the compound as well as other factors known to those of skill in the art.

It is to be noted that dosage values will also vary with the severity of the condition to be treated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered as a single dose, or may be divided into a number of smaller doses to be administered at varying intervals of time.

Topical and other formulations of the Tat-Tyrosinase fusion protein are of utility in enhancing skin or hair pigmentation in humans and other animals. These formulations may be useful for pure cosmetic purposes, simply to obtain a darker skin color for perceived beautification.

The compounds of this invention act primarily by increasing mammalian melanocyte tyrosinase, the rate-limiting enzyme in the production of melanin from tyrosine and DOPA. If desirable these formulations could also be used to increase pigmentation in hair, albeit during the biosynthesis of hair, by enhancing pigment production within the melanocytes of hair follicles. The formulations would likely not affect the already emerged pigmented portions of hair, unlike a coloring agent.

The formulations useful in the present invention contain biologically effective amounts of the Tat-Tyrosinase fusion protein. A biologically effective amount of the active agent is understood by those skilled in the art to mean that a sufficient amount of the agent in the composition is provided such that upon administration to the human or animal by topical route, sufficient active agent is provided on each application to give a desired result. However, the biologically effective amount of the active compound is at a level that it is not toxic to the human or animal during the term of treatment. By a suitable biologically compatible carrier, when the fusion protein is topically applied, it is understood that the carrier may contain any type of suitable excipient in the form of cosmetic compositions, pharmaceutical adjuvants, lotions, creams, and the like. In one embodiment the active agent is administered in a liposomal carrier. The active agent is administered for a sufficient time period to enhance the desired symptoms and the clinical signs associated with the condition being treated, or to achieve the level of desired skin or hair pigmentation. The individual dosage, dosage schedule, and duration of treatment may be determined by clinical evaluations by those of skill in the art.

Solutions or suspensions for topical application can include the following components: a sterile diluent such as water, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediamine-tetraacetic acid (EDTA); buffers such as acetates, citrates or phosphates; and agents for the adjustment of tonicity such as sodium chloride or dextrose. The pH can be adjusted with acids or bases, such as hydrochloric acid or sodium hydroxide.

Suitable vehicles, carriers, or formulations for topical application are known, and include lotions, suspensions, ointments, oil-in-water emulsions, water-in-oil emulsions, creams, gels, tinctures, sprays, powders, pastes, and slow-release transdermal or occlusive patches. Thickening agents, emollients, and stabilizers can be used to prepare topical compositions. Examples of thickening agents include petrolatum, beeswax, xanthan gum, or polyethylene glycol, humectants such as sorbitol, emollients such as mineral oil, lanolin and its derivatives, or squalene. A number of solutions and ointments are commercially available, especially for dermatologic applications.

The fusion proteins can be provided in the form of pharmaceutically-acceptable salts. As used herein, the term “pharmaceutically-acceptable salts or complexes” refers to salts or complexes that retain the desired biological activity of the parent compound and exhibit minimal, if any, undesired toxicological effects. Examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acids, naphthalenedisulfonic acids, and polygalacturonic acid; (b) base addition salts formed with polyvalent metal cations such as zinc, calcium, bismuth, barium, magnesium, aluminum, copper, cobalt, nickel, cadmium, and the like, or with an organic cation formed from N,N-dibenzylethylene-diamine or ethylenediamine; or (c) combinations of (a) and (b); e.g., a zinc tannate salt or the like.

The fusion proteins can be modified in order to enhance their usefulness as pharmaceutical compositions. For example, it is well know in the art that various modifications of the active agent, such as alteration of charge, can affect water and lipid solubility and thus alter the potential for percutaneous absorption. The vehicle, or carrier, can also be modified to enhance cutaneous absorption, enhance the reservoir effect, and minimize potential irritancy or neuropharmacological effects of the composition.

Thus, the present invention provides various formulations of Tat-Tyrosinase and other fusion proteins as topical skin or hair pigment enhancers containing the active agents described above. The present invention further provides formulations as topical anti-oxidants containing the active agent fusion protein and/or functional compounds described above. Such formulations can be made in combination with other active and/or functional ingredients used in skincare products (e.g., organic or inorganic lotion, antioxidant, anti-inflammatory, anti-erythema, antibiotic, antimicrobial, humectant, or other ingredients). Other ingredients can be formulated with the fusion proteins to augment their effect, including but not limited to Vitamin C, Vitamin E, magnesium ascorbyl phosphate, aloe vera extract, and retinoic acids. In addition, alpha-hydroxy acids can be included to speed up the skin pigmentation process by exfoliating surface skin.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

Listed SEQ ID NOs:

| | |
|---|---|
| SEQ ID NO:1: | AAVALLPAVLLALLAP |
| SEQ ID NO:2: | AAVLLPVLLAAP |
| SEQ ID NO:3: | VTVLALGALAGVGVG |
| SEQ ID NO:4: | GALFLGFLGAAGSTMGA |
| SEQ ID NO:5: | MGLGLHLLVLAAALQGAMGLGLHLLLAAALQGA |
| SEQ ID NO:6: | WEAKLAKALAKALAKHLAKALAKALKACEA |
| SEQ ID NO:7: | WEAALAEALAEALAEHLAEALAEALEALAA |
| SEQ ID NO:8: | LARLLARLLARLLRALLRALLRAL |
| SEQ ID NO:9: | KLLKLLLKLWKLLLKLLK |
| SEQ ID NO:10: | RQIKIWFQRRMKKWK |
| SEQ ID NO:11: | YGRKKRRQRRR |
| SEQ ID NO:12: | DAATATRGRSAASRPTERPRAPARSASRPRRPVE |
| SEQ ID NO:13: | GWTLNSAGYLLGKINLKALAALAKKIL |

What is claimed is:

1. A method for the treatment of a skin or hair condition in a mammal, comprising the steps of:
applying to a skin area of said mammal in need of such treatment, a composition comprising an effective amount of a fusion protein and a pharmaceutically acceptable carrier,
wherein said fusion protein comprises a peptide having superoxide dismutase (SOD) activity and a membrane transport sequence.

2. The method of claim 1, wherein said skin condition is skin pigmentation.

3. The method of claim 1, wherein said skin condition is wrinkle.

4. The method of claim 1, wherein said treatment of the skin condition is skin whitening.

5. The method of claim 1, wherein said skin condition is skin burn.

6. The method of claim 5, wherein said skin burn is caused by the over exposure to sunlight.

7. The method of claim 1, wherein said hair condition is hair loss.

8. The method of claim 1, wherein the composition is applied topically.

9. The method of claim 1, wherein said carrier comprises water, glyceryl stearate, cetyl alcohol, propylene glycol stearate, polysorbate 60, sorbitan stearate, Vitamin E, methylparaben, propylparaben, and/or BHA.

10. The method of claim 9, wherein said carrier contains said water (20-80% by weight), said glyceryl stearate (0.25-12% by weight), said cetyl alcohol(0.1-11% by weight), said propylene glycol stearate (0.1-11% by weight), said polysorbate 60 (0.1-5% by weight), said sorbitan stearate (0.05-5% by weight), said Vitamin E (0.02-4% by weight), said methylparaben (0.01-4% by weight), said propylparaben (0.01-4% by weight), and/or said BHA(0.01-4% by weight).

11. The method of claim 1, wherein said membrane transport sequence is selected from selected from the group consisting of SEQ ID NOS:1-13 and a polylysine.

12. The method of claim 11, wherein membrane transport sequence is SEQ ID NO:11

13. A method for skin depigmentation in a mammal, comprising:
applying to a skin area of said mammal in need of depigmentation, a composition comprising an effective amount of a fusion protein and a pharmaceutically acceptable carrier,
wherein said fusion protein comprises a peptide having SOD activity and a membrane transport sequence.

14. The method of claim 13, wherein said membrane transport sequence is a membrane transport sequence from HIV Tat protein.

15. A method for wrinkle removal in a mammal, comprising:
applying to a skin area of said mammal in need of wrinkle removal, a composition comprising an effective amount of a fusion protein and a pharmaceutically acceptable carrier,
wherein said fusion protein comprises a peptide having SOD activity and a membrane transport sequence.

16. The method of claim 15, wherein said membrane transport sequence is a membrane transport sequence from HIV Tat protein.

17. A method for whitening skin in a mammal, comprising:
applying to a skin area of said mammal in need of whitening, a composition comprising an effective amount of a fusion protein and a pharmaceutically acceptable carrier,
wherein said fusion protein comprises a peptide having SOD activity and a membrane transport sequence.

18. The method of claim 17, wherein said membrane transport sequence is a membrane transport sequence from HIV Tat protein.

19. A method for treating the skin bum in a mammal, comprising:
applying to a skin area of said mammal in need of such treatment, a composition comprising an effective amount of a fusion protein and a pharmaceutically acceptable carrier,
wherein said fusion protein comprises a peptide having SOD activity and a membrane transport sequence.

20. The method of claim 19, wherein said membrane transport sequence is a membrane transport sequence from HIV Tat protein.

21. The method of claim 19, wherein said skin bum is caused by over exposure to sunlight.

22. A method for treating hair loss in a mammal, comprising:
applying to a skin area of said mammal in need of such treatment, a composition comprising an effective amount of a fusion protein and a pharmaceutically acceptable carrier,
wherein said fusion protein comprises a peptide having SOD activity and a membrane transport sequence.

23. The method of claim 22, wherein said membrane transport sequence is a membrane transport sequence from HIV Tat protein.

24. A composition for the treatment of a skin or hair condition in a mammal, said composition comprising:
a fusion protein having SOD activity,
and a carrier.

25. The composition of claim 24, wherein said fusion protein comprises a peptide having SOD activity and a membrane transport sequence.

26. The composition of claim 25, wherein said membrane transport sequence is a membrane transport sequence from HIV Tat protein.

27. The composition of claim 24, wherein said carrier comprises water, glyceryl stearate, cetyl alcohol, propylene glycol stearate, polysorbate 60, sorbitan stearate, Vitamin E, methylparaben, propylparaben, and/or BHA.

28. The composition of claim 27, wherein said carrier contains said water (20-80% by weight), said glyceryl stearate (0.25-12% by weight), said cetyl alcohol(0.1-11% by weight), said propylene glycol stearate (0.1-11% by weight), said polysorbate 60 (0.1-5% by weight), said sorbitan stearate (0.05-5% by weight), said Vitamin E (0.02-4% by weight), said methylparaben (0.01-4% by weight), said propylparaben (0.01-4% by weight), and/or said BHA (0.01-4% by weight).


SEQUENCE LISTING (15 sequences)

SEQ ID NO: 1 - protein, 16 aa, Karposi FGF
AAVALLPAVLLALLAP

SEQ ID NO: 2 - protein, 12 aa, Grb2 (SH2 domain)
AAVLLPVLLAAP

SEQ ID NO: 3 - protein, 15 aa, Integrin Beta-3
VTVLALGALAGVGVG

SEQ ID NO: 4 - protein, 17 aa, HIV-1 GP41(1-23)
GALFLGFLGAAGSTMGA

SEQ ID NO: 5 - protein, 33 aa, Caiiman croc. Lg(v) light chain
MGLGLHLLVLAAALQGAMGLGLHLLLAAALQGA

SEQ ID NO: 6 - protein, 30 aa, Influenza HA-2(1-20)
WEAKLAKALAKALAKHLAKALAKALKACEA

SEQ ID NO: 7 - protein, 30 aa, Influenza HA-2 (1-20)
WEAALAEALAEALAEHLAEALAEALEALAA

SEQ ID NO: 8 - protein, 24 aa, Influenza HA-2 (1-20)
LARLLARLLARLLRALLRALLRAL

SEQ ID NO: 9 - protein, 18 aa, Influenza HA-2 (1-20)
KLLKLLLKLWKLLLKLLK

SEQ ID NO: 10 - protein, 15 aa, Antennapedia third helix (43-58)
RQIKIWFQRRMKKWK

SEQ ID NO: 11 - protein, 11 aa, HIV-1 Tat (47-57)
YGRKKRRQRRR

SEQ ID NO: 12 - protein, 34 aa, HSV transcription factor (267-300)
DAATATRGRSAASRPTERPRAPARSASRPRRPVE

SEQ ID NO: 13 - protein, 27 aa, Galanin + Mastoparan
GWTLNSAGYLLGKINLKALAALAKKIL

SEQ ID NO: 14 - protein, 4 aa, Influenza hemagluttinin (HA-2)
GALA

SEQ ID NO: 15 - protein, 4 aa, Influenza hemagluttnin (HA-2)
KALA